(19) United States
(12) Patent Application Publication
(10) Pub. No.: US 2010/0010632 A1
(43) Pub. Date: Jan. 14, 2010

(54) SAND-BLASTING METHOD USING BIOCOMPATIBLE POLYMERS

(75) Inventors: Xavier Bourges, Mogneneins (FR); Chantal Gobin, Vigneux De Bretagne (FR)

Correspondence Address: Pepper Hamilton LLP, 400 Berwyn Park, 899 Cassatt Road, Berwyn, PA, 19312-1183, US

(73) Assignee: BIOMATLANTE, Vigneux de Bretagne (FR)

(21) Appl. No.: 12/442,799

(22) PCT Filed: Sep. 26, 2007
(86) PCT No.: PCT/EP07/60232

§ 371 Date: Mar. 25, 2009

(30) Foreign Application Priority Data
Sep. 26, 2006 (FR) 0608436

(51) Int. Cl. A61F 2/28; A61L 27/14; B24C 1/06; B24C 11/00

(52) U.S. Cl. 623/16.11; 451/39; 623/23.5

(57) ABSTRACT

The invention relates to a method for modifying the surface state of an implant made of polyaryletheretherketone, and for favouring the osteoconduction and osteointegration thereof in bone surgery, that comprises sand-blasting said implant with abrasive particles of calcium phosphate.

The present invention relates to the preparation of biocompatible implants. More particularly, its object is a method for modifying the surface of polymers, either resorbable polymers or not, and used as medical implants. With it, bioactivity and/or anchoring of these implants may be improved at the contact with living tissues of the implantation site.

The invention consists of sand-blasting these implants with resorbable abrasive particles of the phosphocalcium type. The particles may either be left at the surface of the implant, or dissolved with an acid depending on the sought degree of purity of the surface and depending on the biological effect possibly induced by the particles integrated into the surface.

Sand-blasting of medical implants is very developed, both in dental implantology for titanium implants, or in orthopaedic surgery such as hip prosthesis, osteosyntheses.

It was shown that the microgeometry and roughness of the surface of the implants played a predominant role in their integration at the soft or hard tissues. Microroughness has an influence on the mechanical stability of the implants and on their surface energy, and changes their wettability. In a bone medium, the increase in roughness increases the surface area of the titanium implants, therefore the bone contact and the mechanical and anchoring properties of the latter in the same way.

Patent application FR 04 01151 (published under the number 2 865 939) describes the use of an abrasive powder consisting of calcium phosphate, for modifying the surface of metal implants which are then covered with a silanized hydrogel. In this application, the sand-blasting is intended to improve the grafting of the gel to the surface of the metal, which is not in direct contact with the bone cells.

In vitro proliferation and differentiation of osteoblasts (cells at the origin of the extracellular matrix which is subsequently mineralized) are affected by the surface condition of the implant. Sand-blasted surfaces have irregularities which promote osteointegration. The cells cultivated on this kind of surface secrete a larger extracellular matrix, more easily express alkaline phosphatase, and are more easily differentiated into osteoblasts.

The different sand-blasting media used today are high hardness materials such as alumina, silica or titanium oxide. The problem raised by this kind of product lies in the fact that they are difficult to extract from the treated surfaces. Physico-chemical methods which are often delicate to apply, are generally required in order to ensure proper cleaning of the treated surfaces, such as for example the dissolution of the medium with hydrofluoric acid. Particles of these sand-blasting media, but also ionic complexes may remain at the surface, not leaving the latter in a sufficiently clean condition favourable to bone contact. Indeed, silica or alumina particles may cause the formation of a connective and non-mineralized tissue which will generate poor bone contact. Silica particles may also induce phenomena of reaction to foreign bodies and osteolysis of the host or of newly formed bone tissue, before releasing these toxic particles.

With the strongly acid conditions required for complete removal of silica or alumina, it is not possible to resort to sand-blasting on prostheses in polymeric materials, because the latter are more sensitive to acids than metals. Now metal prostheses, such as titanium, have a hardness which does not allow their use in many surgical indications. This is the case for example of fusion cages for vertebral arthrodesis, which sink into the vertebra when they consist of titanium and in certain cases cause impactions.

Parts in plastic materials are currently implanted, but their bioactivity is more or less significant depending on their chemical structure and on the condition of their surface. Most plastic materials are obtained by moulding or extrusion. They generally have extremely smooth surface conditions, i.e. roughnesses of the order of only 0.5 to 0.2 Ra.

Polyaryletheretherketone (PEEK) is an inert polymer currently used for medical anatomic parts. It is however known that it is impossible to grow bone on this material, which does not generate a direct contact with the bone and causes fibroses. Indeed, it does not have an adhering surface for the cells, notably because of its chemical nature. The latter further does not offer many possibilities of modification by a chemical treatment. Thus, PEEK has only been used up to now as a holding part, but never for establishing intimate bone contact.

The authors of the present invention have found the means of solving this problem, by means of a method with which the surface of PEEK may be modified with a sand-blasting medium of phosphocalcium origin, which may then be removed by means of washing with a weak acid or diluted strong acid. Most of the biocompatible plastics are actually degraded by strong acids, such as hydrofluoric acid, which are currently used for dissolving the sand-blasting media of the alumina or silica type.

With the present invention, it is therefore possible to obtain a PEEK implant, the surface of which is both rough and very clean.

More specifically, the invention relates to a method for modifying the surface condition of a polyaryletheretherketone implant, in order to promote its osteoconduction and osteointegration in bone surgery, involving the sand-blasting of said implant by abrasive calcium phosphate particles.

With this method, it is possible for the first time to increase the surface area of the biocompatible polymeric material and therefore increase the contact with the tissues of the implantation site. It also allows better anchoring of this type of materials as well as better bioactivity by recruiting many more cells at their surface.

The sand-blasting medium may advantageously consist of different calcium phosphate particles obtained naturally or by sintering, selected in the list comprising hydroxyapatite (Ca₁₀(PO₄)₆(OH)₂, tricalcium beta-phosphate (β-Ca₃(PO₄)₂), tricalcium alpha-phosphate (α-Ca₃(PO₄)₂), tetracalcium phosphate (Ca₃(PO₄)₂O₂), octacalcium phosphate (Ca₈H₂(PO₄)₆.5H₂O) as well as their mixtures.

Advantageously, the abrasive particles comprise hydroxyapatite and at least one calcium phosphate selected in the list comprising tricalcium beta-phosphate, tricalcium alpha-phosphate, tetracalcium phosphate and octacalcium phosphate

Advantageously, the hydroxyapatite proportion in the mixture is comprised between 60 and 40% by weight.

Still more advantageously, the abrasive particles comprise hydroxyapatite and tricalcium β-phosphate.

Advantageously, the hydroxyapatite/tricalcium β-phosphate ratio is comprised between 2:3 and 1 by weight.

More advantageously, the abrasive particles comprise 85% of hydroxyapatite and 15% of tricalcium β-phosphate by weight.

Still advantageously, the abrasive particles have a size comprised between 150 and 700 μm, more advantageously between 300 and 700 μm.

Advantageously, the abrasive particles have a Vickers hardness comprised between 450 and 1,200, still more advantageously between 500 and 1,000.

Still more advantageously, the abrasive particles comprise 85% of hydroxyapatite and 15% of tricalcium β-phosphate by weight with a size comprised between 300 and 700 μm and a Vickers hardness comprised between 450 and 1,200, preferably equal to 550.

In order to use such particles as a sand-blasting medium for biomedical usage, analyses of the heavy metal contents are necessary and the results should comply with the standards in effect.

Also the particles should have sufficient hardness in order to retain their abrasive power.

The sizes of these abrasive particles may be variable depending on the selected final roughness. Indeed, the smaller the particles, the more the obtained roughness is small and uniform (with a brushing effect). A contrario, particles generate largest roughness and heterogeneity of the relief of the surface.

These calcium phosphates advantageously have a composition identical with that of the bone and their efficiency as bone substitutes has been widely demonstrated.

The predominant advantage of this type of medium is that it is particularly soluble and this very rapidly with weak acids or diluted strong acids.

The residual calcium phosphate particles may be rinsed from the surface of the polymer with an aqueous solution of weak acid or diluted strong acid.

Advantageously, these acid solutions may be nitric acid diluted to 26% or acetic acid diluted to 15%.

These solutions are sufficiently acid for getting rid of calcium phosphate on the whole surface, without however degrading the latter.

The object of the invention is therefore also a method as defined above, which further comprises a subsequent washing step, advantageously with an aqueous acid solution.

Solubility in an acid medium of a sand-blasting medium consisting of 85% of hydroxyapatite and 15% of tricalcium beta phosphate, for which the particle size is comprised between 300 and 700 μm and the Vickers hardness is 550, was investigated. 5 grams of medium were suspended in 100 mL of 15% nitric acid solution. After 8 minutes, the insoluble content was 0.046% on average. Analyses by EDX spectroscopy (Energy Dispersion X-ray spectroscopy) showed that these insoluble materials mainly consist of unidentifiable calcium phosphate and of traces of silicon and magnesium.

Sand-blasting tests based on calcium phosphate, on titanium parts, have shown that no solid residue was present at the surface of the material after cleaning with acid.

In the case when PEEK has been subject to sand-blasting, it is then possible to modify its surface by subjecting it to a plasma type treatment. It may consist of projecting hydroxyapatite or BCP particles heated to a high temperature by means of a plasma torch, in order to cover the implant with a uniform and smooth calcium phosphate layer. By having a very clean sand-blasted surface, i.e. free of any solid element specific to the sand-blasting medium, it is possible to obtain a more efficient final treatment than with a smooth surface.

By smooth or machined surface is meant a surface for which the roughness is less than 0.5 Ra.

Advantageously, it is also possible to leave calcium phosphate particles at the surface of the polymer. This has the effect of promoting osteoconduction of the implanted material.

After the washing step, the implant according to the invention has the advantage of not releasing any PEEK particles.

The abrased implants according to the invention may be used in orthopaedic surgery, as a compound of a joint, as anatomic parts for blood vessels, membrane for soft tissues, membrane for sustaining and guiding healing, osteosynthesis and more particularly in fusion cages for vertebral arthrodeses.

The method according to the invention may advantageously be applied on inserts or interference screws based on polylactic acid.

The method according to the invention may advantageously be applied to screws, plates and any PEEK osteosynthesis used for maxillofacial, traumatological, orthopaedic surgery, and more generally any bone, osteoarticulary or non-calcified tissular surgery.

The in vivo implantations of these materials have shown that bioactivity is higher than that of a machined, i.e. smooth, surface material.

The object of the invention is therefore also an implant for bone surgery in polyaryletheretherketone able to be prepared by the method according to the invention.

The implant according to the invention advantageously has a roughness larger than or equal to 0.5 Ra, advantageously comprised between 0.5 and 10 Ra.

Advantageously, the surface of the implant is very clean, i.e. it is free of calcium phosphate particles and of released polyaryletheretherketone particles.

The following example for applying the invention is given as an illustration and does not have any limiting character.

### EXAMPLE

PEEK implants were subject to the method according to the invention in order to test their stability and their osteointegration: they were sand-blasted with a sand-blasting medium consisting of calcium phosphates, washed and implanted in a bone site in rabbits. These results were compared with those obtained with non-sand-blasted implants.

### Materials and Methods

The PEEK used for these implantations is of the Optima commercial type (Invibio, Lancashire, Great Britain). 40 cylinders with a diameter of 6 mm and a length of 8 mm were made from an extruded rod.

The sand-blasting medium is a biphasic calcium phosphate (BCP: 85% hydroxyapatite, 15% tricalcium beta-phosphate) made by Biomatlante (Vigneux de Bretagne, France). Its Vickers hardness was calculated with a durometer; its value is 510.

20 implants are kept without sand-blasting (smooth surfaces), and 20 are sand-blasted with BCP particles (of 40-80 mesh, i.e. of a size comprised between 178 and 422 μm), and with a sand-blaster of the Clemco PUL 111D type, with a sand-blasting nozzle of diameter 6 mm.

After sand-blasting, the implants are cleaned in a 26% nitric acid solution for 1 hr in an ultrasonic bath, rinsed 3 times with deionized water and then dried in the oven. The PEEK implants are sterilized in the autoclave at 121° C. for 20 minutes.

6 implants were randomly selected in order to measure their roughness by means of a Surftest SJ-301 profilometer (Mitutoyo, Tokyo, Japan).

The cleanliness of the obtained surface was checked with a scanning electron microscope.

The cylinders were then implanted at the femoral epiphysis of New Zealand rabbits. The rabbits were sacrificed by injection after six and twelve weeks. Dual marking with tetracycline was carried out 10 days before sacrificing the animals. The implants are dehydrated and included in resin for observations and analyses; with a microscanner, polarized light microscope, and scanning electron microscope.

### Results

The roughness measurements have shown that the PEEK ex-works had a roughness of 0.3 Ra±0.1, whereas the sand-blasted PEEK had a roughness of 3.2±0.3 Ra. This roughness may even be increased up to 8 Ra. An EDX analysis of the implants did not show the presence of calcium or phosphorus, thereby meaning that the surface was perfectly clean.

After six and twelve weeks, the non-sand-blasted PEEK and the sand-blasted PPEK are properly osteointegrated. No inflammatory reaction related to sand-blasting is observed.

After twelve weeks, the bone density appears to be similar among both groups of implants, and bone remodelling is observed.

However, the bone architecture appears to be different for both groups. Bone regrowth is more intimate at 6 weeks on sand-blasted surfaces than on smooth surfaces (better osteointegration), no doubt related to better stability of the implant. Osteoconduction is more significant at 6 and 12 weeks for the sand-blasted implants. On both types of surfaces, a bone shell covering the implant is observed. The contact of this lamellar bone shell is more intimate with the sand-blasted surface. The bone architecture is also better organized on the sand-blasted surface, which has many more perpendicular bone bridges. Also, an analysis with a microscanner reveals many more artefacts between non-sand-blasted implants and newly formed bone.

1. A method for modifying the surface condition of an implant in polyaryletheretherketone, in order to promote its osteoconduction and its osteointegration in bone surgery, comprising sand-blasting said implant with abrasive calcium phosphate particles.

2. The method according to claim 1, wherein the abrasive calcium phosphate particles are selected from hydroxyapatite, tricalcium β-phosphate, tricalcium α-phosphate, tetracalcium phosphate, and octacalcium phosphate or any mixture thereof.

3. The method according to claim 1, wherein the abrasive calcium phosphate particles comprise hydroxyapatite and tricalcium β-phosphate.

4. The method according to claim 1, wherein the abrasive calcium phosphate particles have a size comprised between 150 and 700 μm.

5. The method according to claim 1, wherein the abrasive calcium phosphate particles have a Vickers hardness comprised between 450 and 1,200.

6. The method according to claim 1, wherein the abrasive calcium phosphate particles comprise 85% of hydroxyapatite and 15% of tricalcium β-phosphate by weight with a size comprised between 300 and 700 μm and a Vickers hardness comprised between 450 and 1,200.

7. The method according to claim 1, wherein it comprises a subsequent washing step.

8. The method according to claim 1, wherein it comprises a subsequent step for modifying the surface of the implant with a plasma type treatment.

9. An implant for bone surgery in polyaryletheretherketone, wherein it has a roughness larger than or equal to 0.5 Ra.

10. The implant according to claim 9, wherein the surface of the implant is free of calcium phosphate particles and of released polyaryletheretherketone particles.

11. The method according to claim 1, wherein the abrasive calcium phosphate particles have a size comprised between 300 and 700 μm.

12. The method according to claim 1, wherein the abrasive calcium phosphate particles have a Vickers hardness comprised between 500 and 1,000.

13. The method according to claim 7, wherein the subsequent washing step is carried out with an aqueous acid solution.

14. The implant according to claim 9, wherein the implant has a roughness between 0.5 and 10 Ra.

15. The implant according to claim 9, wherein the implant is prepared by the method according to claim 1.
